(19) United States
(12) Patent Application Publication
(10) Pub. No.: US 2023/0404418 A1
(43) Pub. Date: Dec. 21, 2023

(54) SYSTEM AND METHOD FOR CARDIOVASCULAR HEALTH MONITORING

(71) Applicant: ATCOR MEDICAL PTY LTD., West Ryde, New South Wales (AU)

(72) Inventors: Jessi E. Johnson, Sunnyvale, CA (US); Oliver Hao-Yuan Shay, San Mateo, CA (US); Elizabeth Anne Gomes, El Dorado Hills, CA (US)

(73) Assignee: ATCOR MEDICAL PTY LTD., West Ryde, New South Wales (AU)

(21) Appl. No.: 18/338,637

(22) Filed: Jun. 21, 2023

Related U.S. Application Data
(60) Provisional application No. 63/366,761, filed on Jun. 21, 2022.

(51) Int. Cl. A61B 5/021; A61B 5/00; G06F 18/2135

(52) U.S. Cl. CPC A61B 5/02141; A61B 5/746; G06F 18/2135; A61B 5/681

(57) ABSTRACT

A wearable health monitoring system includes one or more RF electromagnetic transmitters (30-300 GHz) configured to transmit RF signals towards the skin over the artery of a user, one or more RF electromagnetic receivers configured to receive a reflected RF signals from a target body region on a user. A system has an attachment mechanism and standoff mechanism position the one or more RF transmitters and receivers relative to the target body region on the user at a known stand-off distance. The reflected signals and the transmitted signal are use to create an Intermediate Frequency signal which is used to determine a composite waveform via Principal Component Analysis, or other Dimensionality Reduction technique.

### CROSS-REFERENCE TO RELATED APPLICATION

The present application claims benefit of U.S. Provisional Patent Application No. 63/366,761, filed Jun. 21, 2022, the content of which is incorporated herein by reference in its entirety.

### BACKGROUND

Cardiovascular parameters may be key vital signs used in evaluating patient health. One example of a cardiovascular parameter used in evaluating patient health is blood pressure, which is the pressure of circulating blood against the walls of the blood vessels. The pressure largely results from the pressure generated by the heart to power the circulatory system. Blood pressure may be typically measured using a blood pressure cuff. However, the “white coat” effect may result in inaccuracies in measurement of blood pressure data. Further, blood pressure cuffs may be less comfortable and may be poorly suited to ambulatory blood pressure measurements.

### SUMMARY

The invention pertains to a method for monitoring cardiovascular health using a wearable device. The wearable device is preferably worn on the wrist of a user and includes one or more RF electromagnetic transmitters, each configured to transmit an RF signal in the frequency range of 30-300 GHz towards a target body region on a subject. The wearable device has one or more RF electromagnetic receivers configured to receive reflected RF signals from the target body region. An attachment mechanism, such as a wrist band, and a standoff mechanism positions the one or more RF electromagnetic transmitters and receivers at a known stand-off distance from the target body region. The target body region is skin over an artery in the subject and the overall purpose of the wearable device to measure the arterial blood pressure waveform with sufficient accuracy to generate useful cardiovascular data.

The method involves the step of transmitting one or more RF signals having a frequency in the range of 30-300 GHz towards the target body region of a subject and receiving one or more reflected RF signals corresponding to the reflection of the transmitted one or more RF signals from the target body region. Next, a raw Intermediate Frequency (IF) signal is generated by mixing or comparing the transmitted one or more RF signals and the reflected one or more RF signals. The raw IF signal comprises signal data from one or more channels of the RF electromagnetic transmitter and the RF electromagnetic receiver. A processor, which can be on the wearable device or remote, generates at least one waveform data set based on the raw or modified IF signal. Dimensionality reduction, such as Principal Component Analysis, is used to combine signals from two or more channels to form a composite waveform.

Cardiovascular features of the composite waveform are extracted, and cardiovascular data are determined from the extracted cardiovascular features using a cardiovascular model. The cardiovascular data, or some of the data is displayed on the wearable device.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which

FIG. 1A illustrates a representation of a system, in accordance with some embodiments.

FIG. 1B illustrates a system comprising a remote processing subsystem, in accordance with some embodiments.

FIG. 1C illustrates a representation of a system that depicts electromagnetic signals being reflected from a target region, in accordance with some embodiments.

FIG. 1D illustrates a system comprising a guiding structure, in accordance with some embodiments.

FIG. 2 is a flowchart of an exemplary method for generating cardiovascular data, in accordance with some embodiments.

FIG. 3 is a flowchart of an exemplary process for generating a waveform data set, in accordance with some embodiments.

FIG. 4 is a flowchart of an exemplary method for performing a quality assessment on the raw electromagnetic signals received, in accordance with some embodiments.

FIG. 5 is a flowchart of an exemplary method for performing a quality assessment on the transformed electromagnetic signals received, in accordance with some embodiments.

FIG. 6 is a flowchart of an exemplary method for performing a quality assessment on the initial principal component analysis (PCA) computation performed on the pre-processed signal data, in accordance with some embodiments.

FIG. 7 is a flowchart of an exemplary method for performing a quality assessment on the post-processed waveform data, in accordance with some embodiments.

FIG. 8 shows a computer system that is programmed or otherwise configured to implement methods provided herein.

### DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Certain inventive embodiments herein contemplate numerical ranges. When ranges are present, the ranges include the range endpoints. Additionally, every sub range and value within the range is present as if explicitly written out. The term “about” or “approximately” may mean within an acceptable error range for the particular value, which will depend in part on how the value is measured or determined, e.g., the limitations of the measurement system. For example, “about” may mean within 1 or more than 1 standard deviation, per the practice in the art. Alternatively, “about” may mean a range of up to 20%, up to 10%, up to 5%, or up to 1% of a given value. Where particular values are described in the application and claims, unless otherwise stated the term “about” meaning within an acceptable error range for the particular value may be assumed.

Systems and methods of the present disclosure generally relate to the biometric field, and more specifically to a new and useful system and method for monitoring cardiovascular health.

In some embodiments, systems described herein generate waveform data by processing one or more signal datasets derived from a radiofrequency (RF) system, wherein said waveform data includes features correlating with a cardiovascular parameter for a user, such as blood pressure measurement or other blood pressure data. In some embodiments, systems described herein improve the signal quality of signal datasets derived from the RF system and used in determining cardiovascular parameters. For example, the technology may utilize transformed data which may allow for improved methods of filtering noise signals from the datasets. In some embodiments, systems described herein use principal component analysis (PCA) for processing the signal dataset(s), thereby generating a single composite waveform that can be correlated with cardiovascular parameters. In some embodiments, systems described herein are configured to identify and/or correct for poor signal quality, environmental conditions, motion artifacts, and/or noise. In some embodiments, such identification and/or correction is performed one or more times during the signal processing process between receiving the signals to generating an output (e.g., blood pressure measurement or another cardiovascular measurement). As such, systems described herein are configured to accommodate for variables affecting consistent signal quality, including user variations (e.g., different physiology, different motion, different ways of operating the RF system, etc.), RF system variations (e.g., different orientations of the RF sensor device, different arteries at which measurements are collected, etc.), and/or other variations.

In some embodiments, signal datasets collected at various locations can be used in evaluating body movement-related data (e.g., tissue movement-related data, respiration, heartbeat, arterial motion, stroke volume, pulse parameters such as pulse transit time and pulse wave velocity, etc.), from which cardiovascular parameters (e.g., heart beat metrics, blood pressure metrics, pulse rate metrics, physical activity metrics, metrics correlated with cardiovascular-related health, pulse oximetry metric, arterial metrics, respiration metrics, etc.) may be determined. In some embodiments, the cardiovascular parameters are used in a range of health and fitness applications, such as health monitoring, sports coaching, diagnosis and prediction of certain disease conditions such as cardiovascular related conditions (e.g. hypertension, atherosclerosis, arrhythmia, peripheral artery disease, aortic dissection, blood vessel insufficiency, pulmonary disease) and health-related emergency alerts. In some embodiments, an RF system described herein (e.g., based on RF detection and ranging) may be compact, unobtrusive, and enable continuous monitoring of cardiovascular parameters, overcoming issues of inconvenience, discomfort, lack of adherence, and other issues associated with, for example, a blood pressure cuff. In some embodiments, the RF system can be resilient to variables (e.g., ambient light, presence of tattoos, perspiration at site of measurement, etc.) affecting signal quality for non-cuff based systems.

In some cases, the technology can continuously monitor cardiovascular parameters. Cardiovascular parameter monitoring can additionally or alternatively be dynamically triggered (e.g., in response to detecting an inactive user state based on motion data collected at a motion sensor of the RF system).

### 1. System

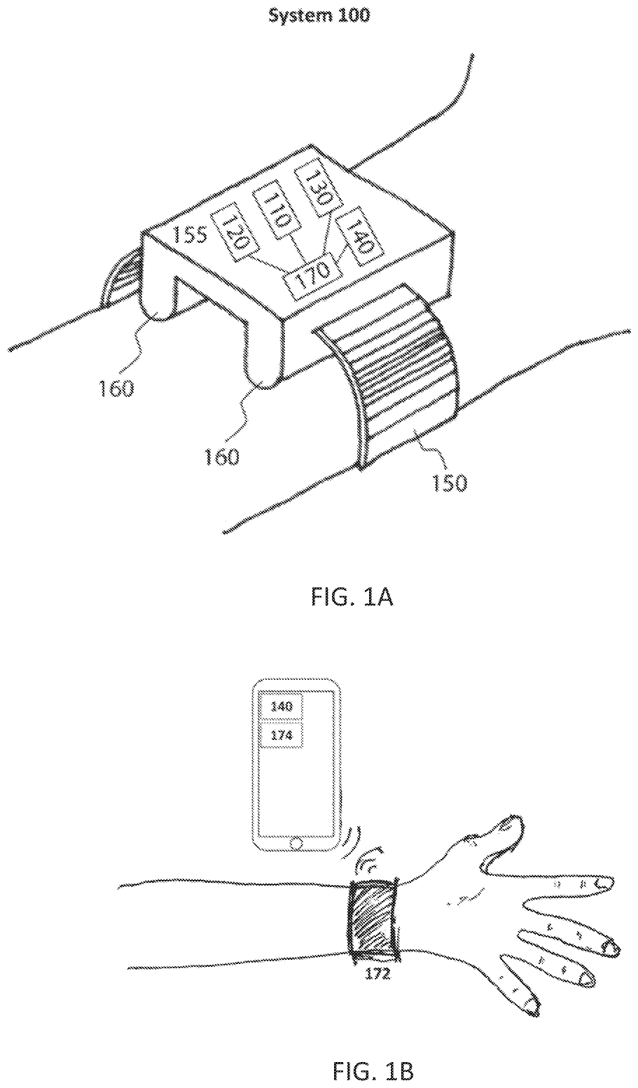

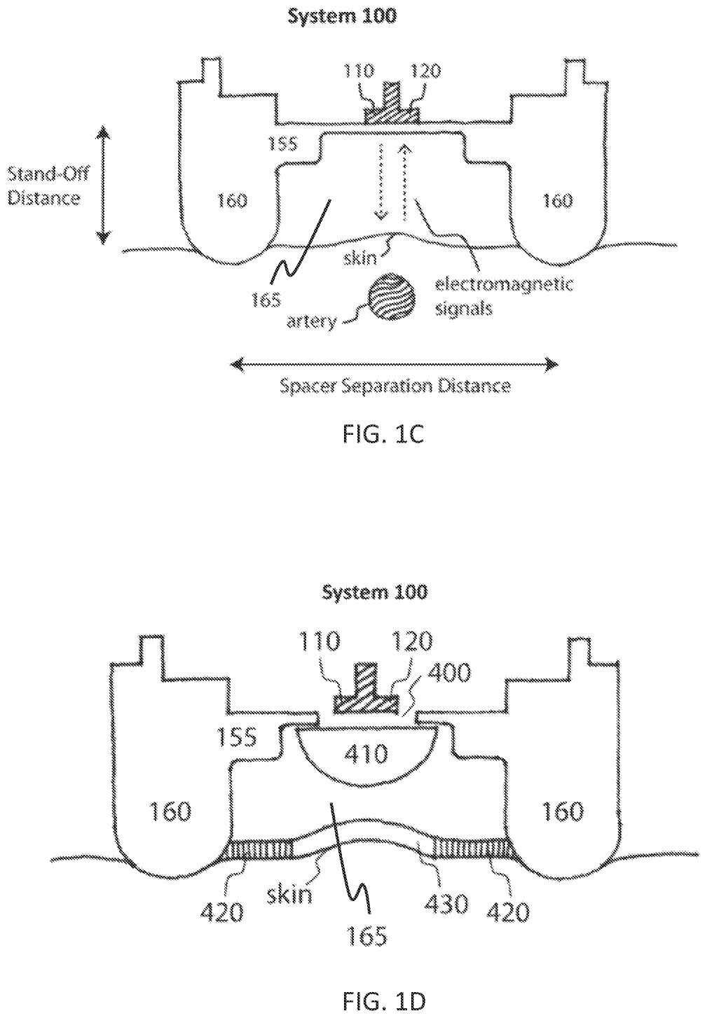

FIGS. 1A-1D show various examples of a system (e.g., RF system) of the present disclosure. FIG. 1A illustrates an exemplary representation of a system 100, in accordance with some embodiments. In some embodiments, the system 100 comprises one or more of: an electromagnetic transmitter 110 and an electromagnetic receiver 120. In some embodiments, the system 100 is a health monitoring system. In some embodiments, the transmitter 110 is configured to transmit an electromagnetic signal towards a region of a user. The region may be a body region. In some embodiments, the electromagnetic receiver 120 is configured to receive reflected energy from a user. As used herein, the term “user”, “subject”, or “patient” may be used interchangeably, and refer to a person for whom a cardiovascular metric (e.g., blood pressure data) is being obtained.

In some embodiments, the system 100 comprises one or more of: a calibration sensor 130 and an activity sensor 140. In some embodiments, the calibration sensor measures reference parameters of a user. In some embodiments, the parameters include cardiovascular-related parameters of a user. In some embodiments, cardiovascular parameters may include one or more of: vessel pressure, stiffness, or motion. In some embodiments, an activity sensor 140 is configured to detect the activity state of a user.

In some embodiments, the system 100 is configured as a wearable device for the user. In some embodiments, the system 100 comprises an attachment mechanism 150. In some embodiments, the attachment mechanism 150 is configured to fix various parts of system 100 to a user. For example, in some embodiments, the attachment mechanism 150 is configured to fix one or more of: the electromagnetic transmitter 110, electromagnetic receiver 120, the calibration sensor 130, or activity sensor 140 to the user. In some embodiments, the attachment mechanism 150 comprises a belt, a strap, a watch band, an adhesive tape, an article of clothing (a sock, a glove, a pants, a shirt, an arm band, an arm warmer, a leg warmer, etc.). In some cases, the attachment mechanism 150 comprises a band. In some cases, the attachment mechanism 150 comprises: Velcro®, straps, adhesive, silicone, and/or any other suitable attachment mechanism or combination thereof. In some cases, the attachment mechanism 150 is configured to attach to a separate device mounted to the user. In some cases, the attachment mechanism 150 is configured to couple the transmitter 110 and receiver 120 to a wearable fitness device. In some cases, the attachment device 150 is fixed to an exercise machine and enables temporary mounting to a user. In some cases, the attachment device 150 is fixed to a hospital bed and enables temporary mounting to a user.

In some embodiments, the system 100 comprises a stand-off mechanism 160 for fixing the electromagnetic transmitter 110 and electromagnetic receiver 120 at a stand-off distance from a target body region on the user. In some embodiments, the stand-off distance is a specified stand-off distance. In some embodiments, the stand-off distance ensures that the target body region remains outside of the reactive near-field of the electromagnetic transmitter 110 and electromagnetic receiver 120. The reactive near-field is a region where electric and/or magnetic fields have large spatial gradients, such that motion of a target body region on a user can cause unexpected and/or distorted radar output. In some embodiments, a boundary of the reactive near-field region can be estimated by the following equation:

$$R = 0.62 * \frac{\overline{{D}^{3}}}{Ls}$$

Where R is the distance from the antenna (e.g., of the system 100) to the near-field boundary, D is the antenna aperture size and L is the wavelength. In some embodiments, the wavelength and antenna aperture result in a reactive near field boundary of 1 mm or less. In some embodiments, the boundary is 2 mm or less.

In some cases, the system 100 comprises a processor 170, which may be electrically coupled and in communication with one or more of: the electromagnetic receiver 120, electromagnetic transmitter 110, calibration sensor 130, and activity sensor 140. In some embodiments, processor 170 is configured to evaluate cardiovascular-related parameters of the user based on the received reflected energy (for example, via the electromagnetic transmitter 110 and receiver 120), including in some cases, transforming and/or processing raw and/or transformed intermediate frequency (IF) data using dimensionality reduction techniques (e.g., principal component analysis (PCA)), as described herein. In some embodiments, system 100 includes other components (not shown), such as a display, touch screen, battery, power charging unit, wireless communication (e.g. Wi-fi, Bluetooth), GPS, and other peripheral units. In some cases, an external processor (e.g. remote server) is used to evaluate cardiovascular-related parameters of the user. In some embodiments, such external processor includes a smart device (e.g., smart phone, tablet, smartwatch), a computer (laptop, desktop), and/or cloud computing.

In some embodiments, the electromagnetic transmitter 110 is configured to generate and transmit electromagnetic signals towards a target body region of the user, such as an arterial surface pulse. The electromagnetic signals may reflect off of the user (e.g., off a target body region of the user). In some embodiments, the reflected signals are received by the electromagnetic receiver 120. As used herein, the term “transmit signal” and “transmitted signal” may be used interchangeably and may refer to a transmitted electromagnetic signal. In some cases, the transmit signal is in the millimeter wave frequency band (e.g. between 30 and 300 GHz). In some cases, the transmit signals are millimeter-wave signals between 57 and 71 GHz. In some cases, the transmit signals are millimeter-wave signals between 58.0 and 63.5 GHz. In some embodiments, the electromagnetic transmitter 110 utilizes frequency modulated continuous wave signals. In some embodiments, frequency modulated continuous wave signals are similar to those used for a frequency modulated continuous wave radar system. In some embodiments, the electromagnetic transmitter 110 utilizes fixed continuous wave signals. In some embodiments, the electromagnetic transmitter 110 utilizes pulsed signals. In some embodiments, the electromagnetic transmitter 110 utilizes step frequency radar signals. In some embodiments, the electromagnetic transmitter 110 utilizes pulsed modulated continuous wave signals. In some embodiments, the electromagnetic transmitter 110 comprises a transmit antenna subsystem for facilitating radiation of the electromagnetic energy.

In some embodiments, the electromagnetic transmitter 110 is a multi-channel transmitter. In some embodiments, the multi-channel transmitter is a 2 channel transmitter. In some embodiments, the multi-channel transmitter has 2, 3, 4, 5, 6, 7, 8, 9, about 10, about 20, about 50, about 100 or more channels.

In some embodiments, the electromagnetic receiver 120 is configured to receive reflected signals from a user. As used herein, the term “reflected signal” and “received signal” may be used interchangeably and may refer to a received electromagnetic signal (e.g., reflected from a user). In some embodiments, the reflected signal comprises an arterial pulse waveform from a target body region of a user. In some embodiments, characteristics of the received signals, such as the timing, shape, phase, amplitude, etc. of the received signals, are affected by the arterial pulse waveform, which may in turn enable the determination of the user's blood pressure and/or other cardiovascular measurements. In some embodiments, the transmitted and/or received signals are communicated to the processor 170. In some embodiments, the received signal is mixed with or compared to the transmitted signal (e.g., a sample of the transmitted signal) to generate and/or form an intermediate frequency (IF) signal that is communicated to the processor 170. In some embodiments, the receiver(s) 120 and transmitter(s) 110 form a radar system. For example, in some embodiments, as described herein, the processor 170 processes the received signal and/or the IF signal to determine the various characteristics of the received signals (as described herein, such as timing, shape, phase, etc.), and/or extract information related to the position and motion of the target, such as a body region of a user. In some embodiments, the radar system may generate the IF signal using the transmitted signal and the received signal. For example, in some cases, one or more analog circuitry of the radar system may generate the IF signal. In some embodiments, the radar system may be a frequency modulated continuous wave radar system. In some embodiments, the radar system comprises a continuous wave radar, such as continuous wave doppler radar. In some embodiments, the radar system comprises a pulsed radar, such as, for example, impulse-radio ultra-wideband radar. In some embodiments, only the received signals are communicated to the processor 170. In some embodiments, the transmitted signals are not communicated to the processor 170. In some embodiments, only the IF signals are communicated to the processor 170. In some embodiments, the electromagnetic receiver 120 comprises a receive antenna subsystem for facilitating reception of electromagnetic energy. In some cases, the receive antenna subsystem may include a downconverter (also referred to as a “measurement circuit” and “mixer”). In some cases, the downconverter is configured to mix the received signal with one or more signals. In some cases, the downconverter can include one or more paths (also referred to as “mixer paths”), where the downconverter is configured to mix the received signal with a signal (e.g., different signal) on each of the one or more paths. In some cases, the downconverter is configured to mix the received signal with a sample of the transmitted signal to generate the IF signal. In some cases, the downconverter can include a first path and a second path, where the first path is configured to mix the received signal with a sample of the transmitted signal (e.g. In-Phase signal) that is aligned in phase with the received signal and the second path is configured to mix the received signal with a sample of the transmitted signal (e.g., Quadrature signal) that is 90-degrees out of phase with the received signal. In some cases, the receive antenna subsystem includes more than one downconverter. In some cases, transmit and receive functions may be performed using the same antenna subsystem.

In some embodiments, the electromagnetic receiver 120 comprises a multi-channel receiver. In some embodiments, the multi-channel receiver is a 2 channel receiver. In some embodiments, the multi-channel receiver has 2, 3, 4, 5, 6, 7, 8, 9, about 10, about 20, about 50, about 100 or more channels. In some embodiments, a channel of the electromagnetic receiver 120 corresponds to a receive antenna. In some embodiments, a channel of the electromagnetic receiver 120 corresponds to a receive antenna communicatively coupled to a particular mixer path of a downconverter. In some cases, if a downconverter includes one or more mixer paths, each mixer path of the downconverter that is communicatively coupled to a receive antenna corresponds to a respective channel of the electromagnetic receiver 120.

In some embodiments, the attachment mechanism 150 is configured to fix the electromagnetic transmitter 110 and electromagnetic receiver 120 to the user. In some embodiments, the attachment mechanism 150 orients the electromagnetic transmitter 110 and electromagnetic receiver 120 towards a target, e.g., a body region. In some embodiments, the body region is a region where arterial pulsing of the user is present on the skin surface. In some embodiments, the system (e.g., system 100) comprises an enclosure 155 to house the electromagnetic transmitter 110 and electromagnetic receiver 120. In some embodiments, the enclosure 155 can removably house and/or removably couple to one or more of: the electromagnetic transmitter 110, electromagnetic receiver 120, the calibration sensor 130, or the activity sensor 140 of the system 100. For example, the enclosure 155 may include a cavity, enclosure, slot, or another suitable physical mechanism configured to removably couple to and/or house one or more of: the electromagnetic transmitter 110, electromagnetic receiver 120, the calibration sensor 130, or the activity sensor 140 of the system 100. In some embodiments, the attachment mechanism 150 comprises the enclosure 155. In some embodiments, the enclosure 155 is removably attachable to the attachment mechanism 150. In some embodiments, the enclosure 155 has geometrical features that allow minimal perturbation of the transmit and/or receive signal. In some embodiments, the enclosure 155 has as a thin plastic or open window. In some embodiments, the enclosure 155 has a second stand-off or cutaway region. In some embodiments, the enclosure 155 has an additional antenna assembly In some embodiments, the attachment mechanism 150 is configured to removably couple one or more of: the electromagnetic transmitter 110, electromagnetic receiver 120, the calibration sensor 130, or activity sensor 140 to the user. For example, the attachment mechanism 150 may removably couple one or more of: the electromagnetic transmitter 110, electromagnetic receiver 120, the calibration sensor 130, or activity sensor 140 to a user via an adhesive.

In some embodiments, the enclosure 155 is configured to attach to a separate device mounted to the user. The separate device (also referred to as an “anchor”) can removably house and/or couple to the system 100. For example, the separate device can removably house or couple to the enclosure 155. In some embodiments, the separate device has geometrical features that allow minimal perturbation of the transmit and/or receive signal. In some embodiments, the separate device has as a thin plastic or open window. In some embodiments, the separate device has a stand-off or cutaway region. As described herein, the separate device may be removably coupled to the user (e.g., via an adhesive).

In some embodiments, the stand-off mechanism 160 is configured to offset the electromagnetic transmitter 110 and electromagnetic receiver 120. In some embodiments, the stand-off mechanism positions the electromagnetic transmitter 110 and/or electromagnetic receiver 120 at a known stand-off distance from a target body region on the user. In some embodiments, the known stand-off position is about 1 millimeter (mm), about 2 mm, about 3 mm, about 4 mm, about 5 mm, about 6 mm, about 7 mm, about 8 mm, about 9 mm, about 10 mm, about 20 mm, about 30 mm, or more. In some embodiments, the stand-off distance is within a range from about 2 mm to about 5 mm, about 1 mm to about 10 mm, about 2 mm to about 7 mm, about 1 mm to about 20 mm, about 4 mm to about 14 mm, etc. In some embodiments, the stand-off is greater than about 2 mm, greater than about 3 mm, or more. In some embodiments, the offset positions the enclosure 155 at a distance (stand-off distance and/or spacer separation distance) such that the enclosure 155 avoids perturbing the physiology of the target body region. In some embodiments, the offset positions the enclosure 155 at a distance such that the enclosure 155 enables the user's skin and tissues overlying the artery to move freely relative to the transmitter 110 and receiver 120. In some embodiments, the offset, via the stand-off distance, maintains the target body region outside of the reactive near-field of the electromagnetic transmitter 110 and electromagnetic receiver 120, thus preventing unpredictable performance.

FIG. 1C illustrates an exemplary representation of a system (e.g., system 100) that depicts electromagnetic signals being reflected from a target region, in accordance with some embodiments. In some embodiments, the stand-off mechanism 160 comprises spacers. For example, there may be two spacers. In some embodiments, the stand-off mechanism 160 comprises three spacers, four spacers, or more. In some embodiments, the stand-off mechanism 160 is defined by a circular or square cut-out from the enclosure 155 (and/or a separate device). However, any suitable stand-off mechanism 160 may be used. In some embodiments, each spacer is sized and shaped to facilitate comfort to the user. For example a spacer may be curved at the location resting against the user (or resting on an object adjacent to the user, such as clothing). In some embodiments, the curved edges also serve as guides for orientation and placement of the attachment mechanism 150 on the user.

In some embodiments, the stand-off mechanism 160 comprises one or more projections that are arranged in a grid. In some embodiments, the projections are rods with rounded ends. In some embodiments, the stand-off mechanism 160 comprises a discrete component of the system (e.g., system 100), or is integrated with one or more other components of the system 100 (e.g., the attachment mechanism 150, the enclosure 155, etc.). In some embodiments, the material on the edges of the stand-off spacers (e.g., 160) comprise an adhesive. In some embodiments, the material on the edges of the stand-off spacers comprise a material that increases friction. In some embodiments, the material on the edges of the stand-off spacers comprise a material that increases comfort. In some embodiments, the stand-off distance prescribed by the stand-off mechanism is between about 1 and about 20 millimeters. In some embodiments, the stand-off distance is between about 4 and about 14 millimeters. In an alternative embodiment, the stand-off distance is distinct for each transmitter 110 and receiver 120 of the system (e.g., system 100).

FIG. 1D illustrates an exemplary system (e.g., system 100) comprising a guiding structure, in accordance with some embodiments. In some embodiments, material is arranged between the user's skin and the transmitter 110 and/or receiver 120, either as part of the system 100 or separate to the system 100. In some cases, the region 165 (for example, as shown in FIGS. 1C-1D) between the electromagnetic transmitter 110 and electromagnetic receiver 120 comprises a guiding structure that enhances the received signal reflected from the target body region. In some cases, the guiding structure comprises a window 400 that is cut or inserted into the enclosure 155 and is substantially transparent to the transmit and receive signal. In some cases, the window 400 can be circular, square, rectangular, or any other suitable shape.

In some embodiments, the window 400 is configured such that less than about 50% of a transmitted signal is reflected by the window 400. In some embodiments, the window 400 is configured such that less than about 10% of a transmitted signal is reflected by the window 400. In some embodiments, the window 400 is configured such that less than about 5% of a transmitted signal is reflected by the window 400. In some embodiments, the window 400 is constructed from low-loss materials such as air, polystyrene, polycarbonate, polyimide or other such materials. In some embodiments, the stand-off mechanism 160 and the window 400 may be the same.

In some embodiments, the guiding structure comprises a shaping element 410 that sits in close proximity to the transmit and receive antennas. In some embodiments, shaping element 410 enhances the radiation pattern of the antennas to improve the response from the target body region. In some embodiments, the shaping element 410 is constructed from low-loss insulative materials. In some embodiments, the shaping element 410 is constructed from metallic materials. In some embodiments, the shaping element 410 is constructed from absorptive materials.

In some embodiments, the guiding structure comprises a patch 430 (e.g., an adhesive patch, pigment, removable tattoo, or any suitable type of patch) that is placed on the target body region. In some embodiments, the patch 430 is a reflective patch that is utilized to increase the amount of reflection or scattering of signals from the target (e.g., target region of the body). In some embodiments, the patch 430 is a reflective patch constructed from conductors, such as, for example, copper, gold, or other such materials. In some embodiments, the patch 430 is an absorptive patch that is utilized to decrease the amount of reflection or scattering of the signal from the target (e.g., target region of the body). In some embodiments, the patch 430 comprises both reflective and absorptive portions. For example, in some embodiments, a patch 430 with both absorptive and reflective regions functions as a mask that increases the amount of reflection or scattering of signals from the target at reflective portions of the patch, and decreases the amount of reflection or scattering of signals from the target at absorptive portions of the patch.

In some embodiments, the guiding structure comprises a mask 420. For example, in some embodiments, the guiding structure comprises a mask 420 that is placed around the target body region. In some embodiments, the mask is utilized to reduce the amount of signal acquired from regions outside of the target body region. In some embodiments, the mask is utilized to enhance and/or increase the amount of signal acquired (e.g., reflected) from the target body region. In some embodiments, the mask is constructed from an absorptive or reflective material. In some embodiments, the enclosure itself 155 may form the guiding structure by choosing an appropriate shape and materials for the enclosure.

In some embodiments, as described herein, the processor 170 is used along with the transmitted and received signals to create a radar system (e.g., as part of system 100). In some embodiments, the processor 170 is configured to cause (e.g., trigger) the output of signals from the electromagnetic transmitter 110. In some embodiments, the processor 170 is configured to measure signals received by the electromagnetic receiver 120 or measure IF signals from a downconverter (e.g., a mixer) and, optionally, perform pulse waveform analysis to derive the blood pressure and/or other cardiovascular parameters of the user.

In some embodiments, the processor 170 is used with the transmitted and received signals to create a radar system (e.g., as part of system 100). In some embodiments, measurements from the radar system are used to generate a cardiovascular (e.g., blood pressure) dataset based on the radar data (obtained from, for example the transmitted and received electromagnetic signals). In some embodiments, the processor 170 is configured to process the received signals or the IF signals to generate radar data from the radar system. In some embodiments, the radar data is generated periodically, for example, every about 5 milliseconds (ms), about 10 ms, about 20 ms, about 50 ms, about 100 ms, about 200 ms, about 500 ms, about 1 second (s), about 2 s, about 5 s, about 10 s, about 30 s, about 1 minute (min), about 2 min, about 5 min, about 10 min, about 30 min, about 1 hour (hr), about 2 hr, about 5 hr, about once per day, or rarer. In some embodiments, the processor 170 generates radar data from the radar system over a periodic frequency described herein while the device is being worn. In some embodiments, the radar data is generated at a periodic frequency described herein while the device is in an enabled mode.

FIG. 1B illustrates an exemplary system comprising a remote processing subsystem, in accordance with some embodiments. In some embodiments, the processor 170 comprises a local microprocessor subsystem coupled to the attachment mechanism 150 or the housing 155. In some embodiments, the processor 170 comprises a local processing subsystem 172 as well as a remote processing subsystem 174. In some embodiments, both subsystems comprise a communication system, which are communicatively coupled to one another. In some embodiments, the remote processing subsystem 174 is housed separately from the local processing subsystem 172. In some embodiments, this remote processing subsystem 174 is housed on a phone (e.g., smartphone), tablet, smart watch, computer, cloud computing server, any other suitable device with processing capabilities, or a combination thereof. In some embodiments, the communication system (which may be a part of the processor 170) in any embodiment comprises one or more radios or any other suitable component. In some embodiments, the communication system comprises a long-range communication system, a short-range communication system, or any other suitable communication system. In some embodiments, the communication system facilitates wired and/or wireless communication. Examples of the communication system include: 802.11x, Wi-Fi, Wi-Max, WLAN, NFC, RFID, Bluetooth, Bluetooth Low Energy, BLE long range, ZigBee, cellular telecommunications (e.g., 4G, 5G, 6G, LTE, etc.), radio (RF), microwave, IR, audio, optical, wired connection (e.g., USB), or any other suitable communication device or combination thereof.

In some embodiments, the processor 170 is optionally communicatively coupled to additional sensors, including: electrocardiography sensor, heart rate monitor, photoplethysmography sensor, temperature sensor, blood pressure meter, laser sensor, motion sensor/accelerometer or any other suitable sensor or combination thereof. In some embodiments, an additional sensor may be a supplemental sensor module, as disclosed in U.S. Publication No. 2020/0222011, which is incorporated by reference herein in its entirety. In some embodiments, the processor 170 is powered by a power supply. The power supply may be a wired connection, wireless connection (e.g., inductive charger, RFID charging, etc.), a battery (e.g., secondary or rechargeable battery, primary battery, etc.), energy harvesting system (e.g., solar cells, piezoelectric systems, pyroelectrics, thermoelectrics, etc.), or any other suitable system or combination thereof.

In some embodiments, a display is optionally included with the system (e.g., system 100) to display the contextualized cardiovascular (e.g., blood pressure) dataset and/or to provide alerts (as described herein). In some embodiments, the display is in operative communication with the processor 170. In some embodiments, the display is configured to label blood pressure measurements in accordance with the corresponding detected activity state. In some embodiments, the display plots the blood pressure and the activity state. In some embodiments, the display displays only blood pressure measurements taken during certain activity states. In some embodiments, the display displays only blood pressure measurements taken when the activity state was categorized as ‘at rest.’ In some embodiments, the display is provided with a phone, tablet, or computer, as described herein. In some embodiments, the display is coupled to the attachment mechanism 150, enclosure 155, and/or or stand-off mechanism 160.

### 2. Method

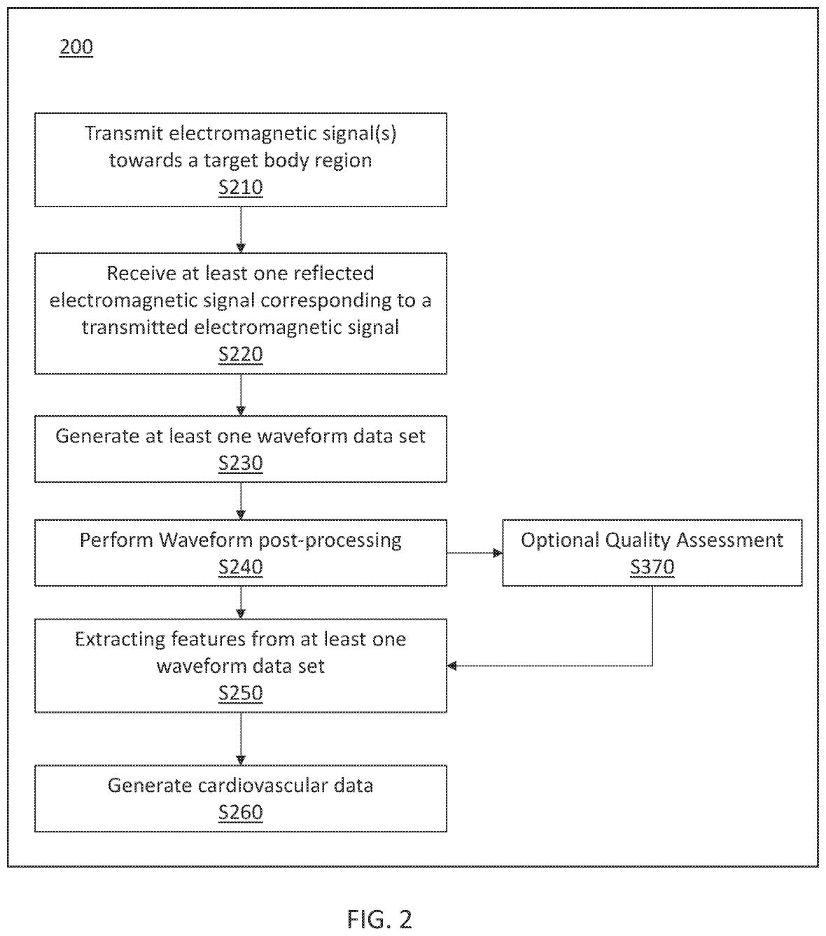

FIG. 2 is an exemplary flowchart of a method 200, in accordance with some embodiments for generating cardiovascular (e.g., blood pressure) data and thereby determining a user's blood pressure and/or other cardiovascular metrics. In some embodiments, the blood pressure and/or other cardiovascular metrics are determined based on radar data acquired using a radar system described herein (e.g., as part of system 100). In some embodiments, the radar data is obtained at any periodicity described herein. In some embodiments, the blood pressure and/or other cardiovascular metrics are determined with no or minimal perturbation of the body. For example, in some embodiments, the blood pressure data is determined with less applied pressure about a target body region of the user than a standard blood pressure cuff. In some embodiments, cardiovascular (e.g., blood pressure) data is determined without applying pneumatic pressure to a patient tissue. Accordingly, in some embodiments, the cardiovascular data (e.g., blood pressure) is obtained through a sufficiently unobtrusive means such that a user may move their arm freely. In some embodiments, the method 200 is implemented by the system 100 (as described herein). In some embodiments, the method 200 is implemented by any system capable of interfacing with a radio frequency receiver (e.g., 120).

As shown in FIG. 2, the method 200 may comprise one or more of: transmitting a plurality of electromagnetic signals directed towards a target body region (e.g., an artery, etc.) of the user via an electromagnetic transmitter (e.g., 110) S210; receiving, at an electromagnetic receiver (e.g., 120), a reflected electromagnetic signal corresponding to at least one of the transmitted electromagnetic signals S220 (e.g., a reflection of at least one of the transmitted electromagnetic signals from the target body region); generating at least one waveform data set by using at least one received reflected electromagnetic signal S230; optionally performing post-processing on the generated at least one waveform data set S240; extracting features from the at least one waveform data set S250; and generating cardiovascular data (e.g., blood pressure data) S260 by processing the extracted features by using a cardiovascular (e.g., blood pressure) prediction model. In some embodiments, the cardiovascular prediction model is used to determine which and/or a type of the features to extract from the at least one waveform data set (e.g., at S250). In some embodiments, the method 200 comprises i) generating training data that includes features extracted from waveform data sets generated from received reflected electromagnetic signals from at least one user; and ii) training the cardiovascular (e.g., blood pressure) prediction model by using the generated training data. As used herein, the terms “waveform data” and “waveform data set” may be used interchangeably.

In some embodiments, at least one component of the health monitoring system 100 performs at least a portion of the method 200.

In some embodiments, transmitting a plurality of electromagnetic signals S210 comprises transmitting signals that reflect off of the skin of the user and are then received at S220. In some cases, the received signals are enhanced via guiding structures such as a window (e.g., 400) or shaping element (e.g., 410), as described herein, to maximize transmission of energy between the transmit and receive antennas (e.g., of the transmitter 110 and receiver 120 respectively) and the target body region.

In some embodiments, as described herein, the guiding structure comprises a material (e.g., reflective patch, partially reflective patch, absorptive patch, pigment, washable tattoo, etc.) fixed to the skin of the user, where the material preferably moves in correlation with movement of the artery. In some embodiments, as described herein, the guiding structure comprises a reflective material that has properties (e.g., weight, thickness, structure, elasticity, flexibility, etc.) that enable the guiding structure to: reflect or scatter incident energy from a transmit antenna back to a receive antenna; and move in correlation with movement of an artery. In some embodiments, as described herein, the guiding structure comprises an absorptive material that has properties (e.g., weight, thickness, structure, elasticity, flexibility, etc.) that enable the guiding structure to absorb incident energy from a transmit antenna, and reduce reflection of the incident energy from the transmit antenna back to a receive antenna. In some embodiments of the absorptive material, the absorptive material has properties that enable the guiding structure to move in correlation with movement of an artery.

In some embodiments, the guiding structure comprises at least one reflective material (as described herein) and at least one absorptive material (as described herein).

In some embodiments, the guiding structure comprises a material (e.g., mask) fixed to the skin of the user, where the material leaves an exposed target body region such as an artery of the user and absorbs energy that strikes other regions (as described herein). In some embodiments, the guiding structure comprises a reflective material that freely moves along with the target body region but does not change its shape during a pulse cycle, thus allowing a target with a more consistent radar cross-section.

In some embodiments, transmitting a plurality of transmitted electromagnetic signals S210 comprises transmitting multiple signals simultaneously using multiple channels of the transmitter 110.

In some embodiments, S210 comprises transmitting millimeter-wave signals. In some embodiments, the electromagnetic transmitter 110 transmits signals with a frequency of 30 to 300 GHz. In some embodiments, the transmitted signals have a frequency between 57 and 71 GHz. In some embodiments, the transmitted signals have a frequency between 58.0 and 63.5 GHz. In some embodiments, the transmitted signals have a frequency between 116 to 123 GHz. In some embodiments, the transmitted signals have a frequency between 122 to 123 GHz. In some embodiments, the transmitted signals have a frequency of approximately 120 GHz. In some embodiments, S210 comprises transmitting frequency modulated continuous wave signals that may be used for frequency modulated continuous wave radar. In some embodiments, S210 comprises transmitting continuous wave signals. In some embodiments, S210 comprises transmitting pulsed signals.

In some embodiments, receiving a reflected electromagnetic signal S220 comprises receiving signals generated by the electromagnetic transmitter (e.g., 110) and reflected off of the user (e.g., at the target body region). In some embodiments, one or more of the timing, shape, phase, and/or amplitude of the reflected electromagnetic signal may be affected by the arterial pulse waveform, which may in turn enable the determination of the user's blood pressure and/or other cardiovascular metrics. In some embodiments, the reflected electromagnetic signal is a frequency modulated continuous wave signal. In some embodiments, the reflected electromagnetic signal is a continuous wave signal. In some embodiments, the reflected electromagnetic signal is a pulsed signal. In some embodiments, the received electromagnetic signal is mixed with the transmitted electromagnetic signal (e.g., a sample of the transmitted electromagnetic signal) to generate and/or form an intermediate frequency (IF) signal, to allow measurement of the transmitted and reflected electromagnetic signal at a frequency lower than the transmitted electromagnetic signal. In some cases, the reflected electromagnetic signal and/or the IF signal is sampled at S220.

In some embodiments, receiving a reflected electromagnetic signal S220 comprises receiving multiple signals simultaneously using multiple channels of the receiver 120.

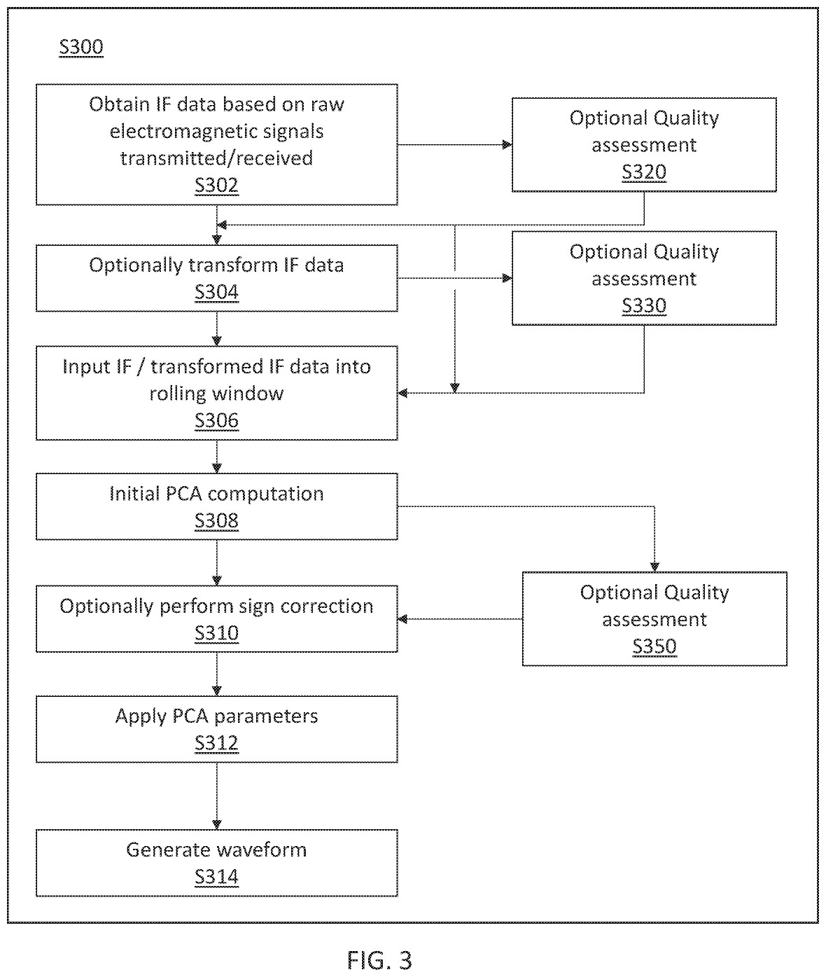

FIG. 3 is an exemplary flowchart of a method for generating a waveform data set S300, in accordance with some embodiments. In some embodiments, the method for generating a waveform data set S300 corresponds to generating at least one waveform data set S230. In some embodiments, generating a waveform data set S300 by using at least one received reflected electromagnetic signal (e.g., received at S220) comprises mixing or comparing the received reflected electromagnetic signal with the transmit signal (e.g., a sample of the transmit signal) to generate and/or form an Intermediate Frequency (IF) signal S302. In some embodiments, the system (e.g., system 100) mixes or compares the received reflected electromagnetic signal with the transmitted electromagnetic signal (e.g., a sample of the transmitted electromagnetic signal) to form an IF signal in the time-domain. As used herein, term “IF signal”, “IF data”, and “IF signal data” may be used interchangeably. In some embodiments, the IF signal comprises information about the timing, shape, phase, envelope, offset and amplitude of the signal that is reflected from the target (e.g., target body region). In some embodiments, the raw IF signal is used as input, for example, to a dimensionality reduction technique such as principal component analysis (PCA), when generating the waveform data. As described with respect to the system 100, generating the IF signal may include mixing or comparing multiple transmitted electromagnetic signals (e.g., samples of multiple transmitted electromagnetic signals) corresponding to multiple channels of the transmitter 110 with multiple reflected electromagnetic signals corresponding to multiple channels of the receiver 120.

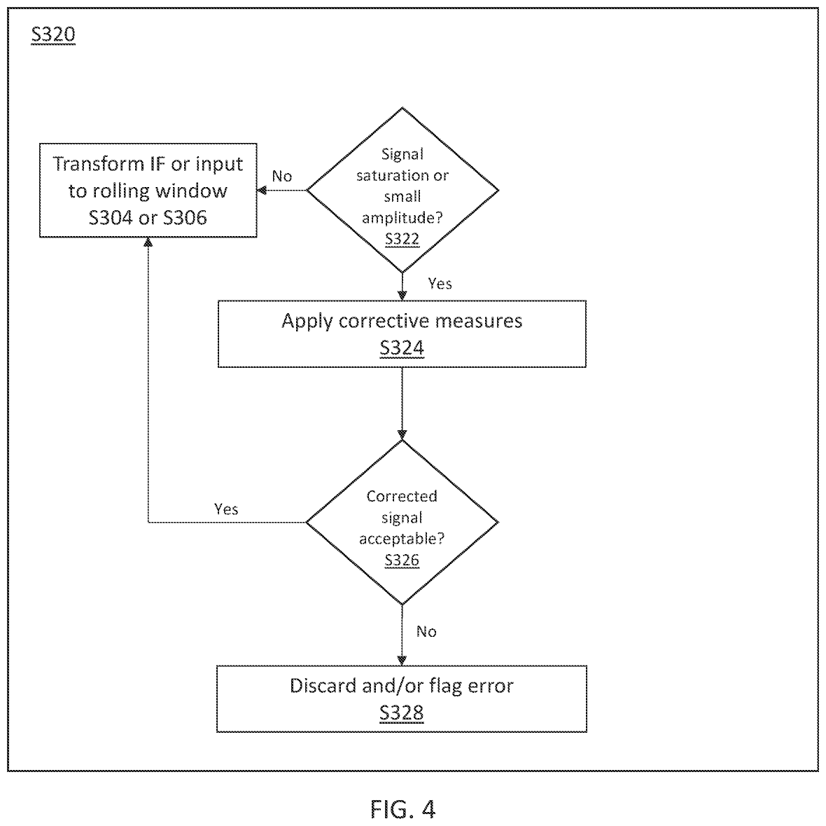

In some embodiments, the system (e.g., system 100) performs a quality assessment S320 on the raw IF signal to ensure low or poor quality IF signals are corrected or discarded. In some embodiments, said quality assessment is performed by the processor 170 of the system 100. FIG. 4 depicts an exemplary flow chart of performing a quality assessment on the raw IF signal data. First, the system detects S322 whether any abnormalities are present with the raw IF signal data. Such abnormalities include, for example, signal saturation with the IF signal data (e.g., any large signals), whether the IF signals are weak and/or noisy, and/or whether the IF signal amplitude is too small. In some embodiments, other examples of abnormalities correlating with poor signal quality include power spectrum analysis and signal-to-noise ratio. If no abnormalities are detected by the system (e.g., via processor 170), the waveform generation process will continue to either transform the IF data S304 and/or input the data into a rolling window S306 (as described herein) prior to serving as input to a principal component analysis. By contrast, if the IF signal data is detected to have one or more abnormalities, such as signal saturation and/or having small amplitude, the system (e.g., via processor 170 of system 100) will apply corrective measures S324 to address such abnormalities. For example, the system, via for example the processor 170, may reduce or increase Tx power (strength of the signal transmitted by transmitter), and/or adjust IF gain/filter settings. In some cases, the amplitude of the signal should be a maximum of ⅔ or 80% of the overall signal range, to help reduce the occurrence of the signal amplitude exceeding said range and skewing the waveform data generated. By contrast, in some cases, the amplitude of the signal should be a minimum of at least about 5% to about 30% of the signal range, so as to help reduce the impact of noise with the received signal skewing the IF signal data for waveform generation. In some embodiments, the corrective measures S324 are applied in real-time, thereby correcting the raw IF signals as they are received. In some embodiments, the corrective measures S324, is applied as a feedback for corrective measures to be applied to subsequent received IF data.

The system may then perform a quality assessment S326 on the corrected IF signal (either the real-time correction or a subsequently received signal through feedback correction), wherein if the corrected IF signal is acceptable (e.g., no presence of signal saturation, weak signal, noisy signal, or a small amplitude), the corrected IF signal then proceeds to either be transformed S304 and/or placed into a rolling window S306. If however, the corrected IF signal is still not acceptable, the corrected IF signal is then discarded and/or the error is alerted to the user S328. For example, in some embodiments, the error is alerted the system via the processor 170, as described herein, and optionally using a display in communication with the processor (as described herein). In some embodiments, as described herein, the system includes a wearable device having a display in operative communication with the processor 170, and configured to display and/or alert the user or other operator regarding poor quality signals received. In some embodiments, the processor 170 is configured to communicate with an external device, such as a smart device (e.g., smart phone), or other device (e.g., a bedside monitor), relating to poor signal quality, wherein said external device is configured to alert the user or other operator. In some embodiments, the quality assessment S320 occurs as a pre-tuning measure, to configure the transmitter and/or receivers so as to obtain proper radar signals for waveform data generation. In some embodiments, the system perform the quality assessment S320 continuously or at least intermittently while generating waveform data, wherein applying corrective measures S324 is part of a feedback for adjusting subsequent signal properties and/or through real-time correction.

In some embodiments, the IF signal (e.g., raw IF signal or corrected IF signal) is transformed S304 to magnitude and phase data, real and imaginary (I/Q) data, or others known in the art. One example of signal transformation is decomposition of the signal. In some embodiments, the IF signal is transformed to the frequency domain for spatial localization of the signal of interest. In some embodiments, a Fast Fourier Transform (FFT) is used to transform (e.g., decompose) each sample of an IF signal (also referred to as “IF signal sample”) into a frequency-domain signal. In some cases, a windowing function may be applied to the IF signal to reduce effects of spectral leakage before applying the Fast Fourier Transform (for example, a Hanning window, a Gaussian, a square function, etc.). One or more additional (or alternate) transforms may be applied to generate more accurate data from the IF signal by, for example, better resolving the true frequency of the IF signal. This may be beneficial for the case when the target (target body region) is in very close proximity to the radar system. In some embodiments, each IF signal sample may be zero-filled before being transformed by applying the FFT. In some embodiments, the FFT may be modified to expand the FFT matrix W to increase resolution, and a 1-norm constraint may be added on the coefficients of the matrix. In some embodiments, the FFT may be modified to expand the FFT matrix W to increase resolution, and a 1-norm solution may be used to determine coefficients in the frequency domain (e.g., as disclosed in Candes, et al. “L1-magic: Recovery of Sparse Signals via Convex Programming”, the contents of which is hereby incorporated by reference).

In some embodiments, an Amplitude Spectrum Capon (ASC) estimator is used to decompose each IF signal sample.

In some embodiments, the system is pre-configured to either use raw electromagnetic signals (radar signals) for generating waveform data (e.g., raw IF signal) or transform the raw electromagnetic signals (as described herein, such as transformed IF signal).

In some embodiments, the transformed IF signal contains any suitable set of one or more frequencies. In such cases, bin selection may be performed to select a frequency bin that is most closely associated with a known distance (e.g., 2-5 mm, any distance disclose herein, etc.) between the target body region and a radar system that may comprise the electromagnetic transmitter (e.g., 110) and the electromagnetic receiver (e.g., 120). In some embodiments, the selected frequency bin represents a range of frequencies (e.g., 25 kHz-125 kHz, etc.).

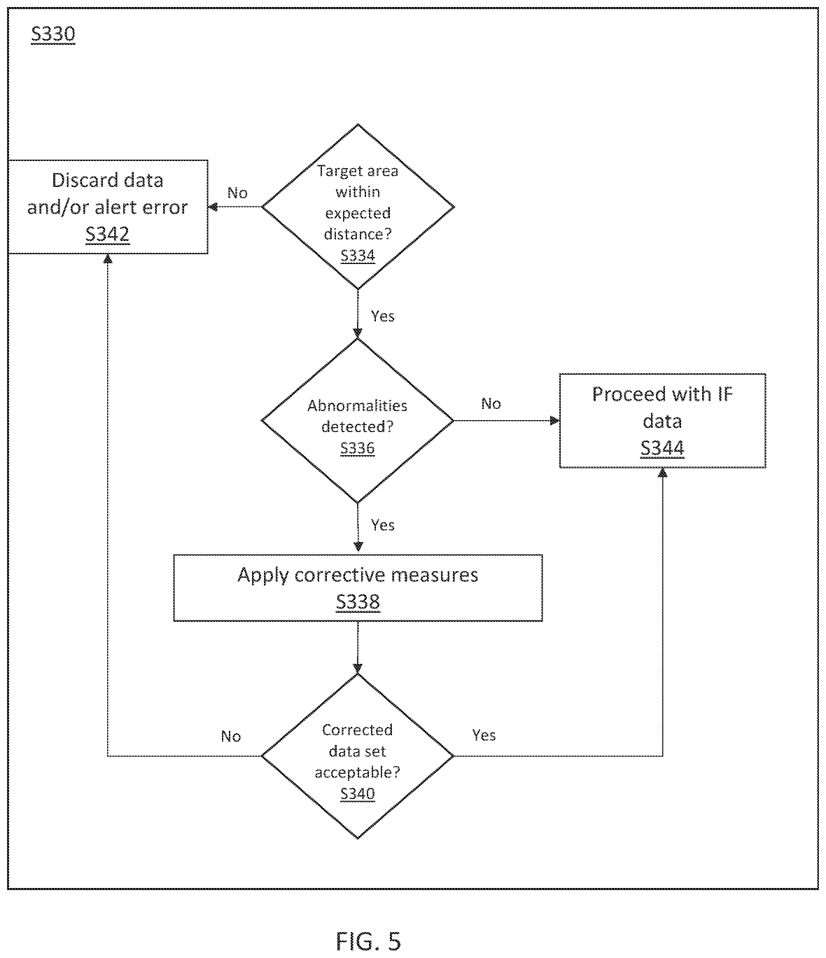

In some embodiments, the system performs a quality assessment S330 on the transformed IF signal to ensure low or poor quality transformed IF signals are corrected or discarded. FIG. 5 depicts an exemplary flow chart of performing a quality assessment on the transformed IF signal data. First, the system (e.g., via processor 170) detects S334 whether the target body region of the subject (e.g., skin surface) is within an expected distance from the transmitter 110, for example. For example, the system may use a range plot to correlate the distance as measured against the transformed IF signal data. If the transformed IF signal data correlates with a target body (e.g., skin surface) that is outside an expected distance range, the transformed IF signal data is then discarded and/or the error is alerted to a user S342 (e.g., via a processor 170 as described herein). The user, or operator, may then adjust the location of the transmitter(s) 110 and/or receiver(s) 120 to ensure the target body region is within the expected distance range.

If, however, the transformed IF signal data correlates that the target area is in the expected distance range, the system (e.g., via processor 170) then optionally performs a quality assessment S336 on the transformed IF signal data, to detect any abnormalities with the transformed IF data. For example, in some embodiments, such abnormalities include detection of an abnormal drift with the transformed IF signal data, as described herein. If abnormalities with the transformed IF signal data are detected, the system (e.g., via processor 170) may then apply corrective measures S338 (e.g., via the processor 170, as described herein), such as performing DC offset/drift correction to address abnormal drift for example. If the corrected transformed IF signal is acceptable, then the system proceeds S344 with the corrected transformed IF signal data for waveform generation. If however, the corrected transformed IF signal is still not acceptable, the corrected transformed IF signal is then discarded and/or the error is alerted to the user S342. For example, in some embodiments, the error is alerted the system via the processor 170, as described herein, and optionally using a display in communication with the processor (as described herein). In some embodiments, as described herein, the system includes a wearable device having a display in operative communication with the processor 170, and configured to display and/or alert the user or other operator regarding poor quality signals received. In some embodiments, the processor 170 is configured to communicate with an external device, such as a smart device (e.g., smart phone), or other device (e.g., a bedside monitor), relating to poor signal quality, wherein said external device is configured to alert the user or other operator. As described herein, in some embodiments, the corrective measures are applied for a subsequent set of IF signal data, such that the corrected transformed IF signal data refers to a subsequently received and transformed set of IF signal data (in comparison with the IF data received from S304, such that said applying corrective measures is part of a feedback operation of the system. In some embodiments, the corrected IF signal refers to the transformed IF data received from S304 such that said corrective measures (S338) are applied in real-time.

With reference to FIG. 3, in some embodiments, a principal component analysis (PCA) is performed on pre-processed signal data (e.g., raw IF signal, transformed IF signal, etc.) to generate data points for a waveform data set. As used herein, the terms “pre-processed signal data” and “pre-processed signals” may be used interchangeably and may refer to any suitable radar data, received signal data, and/or IF signal data (e.g., that is received or processed before S308). In some embodiments, the pre-processed signal data are partitioned within sample windows, such as for example a rolling window. Sample windows may be overlapping and/or non-overlapping. In some embodiments, rolling windows (e.g., consecutive rolling windows) may be overlapping. In some cases, consecutive rolling windows may overlap by a duration (e.g., a preconfigured duration). In some cases, consecutive rolling windows may each overlap by a single frame (e.g., period) corresponding to a frame-rate (e.g., frequency), where the frame-rate corresponds to the acquisition time of the system (e.g. an overlap of 10 msec for a frame rate of 100 Hz). In some cases, consecutive rolling windows may overlap by approximately 4 seconds. In some cases, consecutive rolling windows may overlap according to the heart rate (i.e. they may overlap by a single heart-beat or multiples of the heart-beat). Accordingly, in some embodiments, the pre-processed signals are provided (for example, as input to a PCA as described herein) according to rolling window sizes, which may be defined temporally, according to a pulse rate, or other parameters as known in the art. In some embodiments, the rolling windows are defined temporally, according to a prescribed duration, wherein each successive window includes the pre-processed signals during the given duration. For example, if the rolling window size is set as 5 seconds, then the pre-processed signals are provided as a set of signals obtained in successive and/or overlapping 5 second intervals throughout the duration of a monitoring period by the system 100 (e.g., such as successive and/or overlapping 5 second snapshots of the pre-processed signals). In some embodiments, the temporal rolling window size is from about 0.5 seconds to about 15 seconds. In some embodiments, the temporal rolling window size is from about 0.5 seconds to about 1.5 seconds, from about 3 seconds to about 8 seconds, from about 5 seconds to about 10 seconds, at least about 0.5 second, or at most about 15 seconds. In some embodiments, the rolling windows are defined by pulse rate, such that pre-processed signals are placed in windows according to successive pulses or groups of pulses by a subject.

In some embodiments, the rolling window size parameter are defined by a user, for e.g., via the processor 170. In some embodiments, the rolling window size is fixed. In some embodiments, as described herein, the rolling window size parameters are adjusted based on the generated waveform, to account for fluctuations, such as changes to heartbeat, etc. For example, in some embodiments, the rolling window is adjusted (e.g., dynamically adjusted, adjusted off-line, etc.). In some embodiments, results from waveform data generation are used to inform selection of future sample windows. However, the size of each sample window may be determined in any suitable manner. For example, as described herein, in some embodiments, a quality assessment of the initial PCA computation is performed S350, which in some cases, applies corrective measures that include resizing a rolling window size.

In some embodiments, a small rolling window size is selected, which correlates to less computational power used by the system 100. In some embodiments, if the large fluctuations with the signal data are detected (e.g., via the waveform generated, the initial PCA computation, etc.), the rolling window size may be increased.

In some embodiments, receiving a reflected electromagnetic signal at S220 comprises receiving multiple signals simultaneously using multiple channels (e.g., multiple antennas and/or an antenna with multiple mixer paths such as an In-Phase and Quadrature path) of the receiver 120. In some cases, three channels of data are received at S220, and pre-processed signal data (e.g., raw IF signal data and/or transformed IF data) for each of the three channels is available for use in generating the waveform data. In some embodiments, data from all three channels is combined to generate the waveform data. In some embodiments, principal component analysis (PCA) is used to combine multiple received pre-processed signal datasets into a single waveform. In some embodiments, PCA is utilized to combine radar data (e.g., pre-processed signals as described herein) into a composite waveform, wherein said pre- processed signals data are used as an input into a PCA function. For example, in some embodiments, the phase and/or magnitude of the FFT of the raw IF signal (for example) is utilized as an input into a PCA function. In some cases, the real and imaginary components of the FFT (i.e. I/Q data) can also be utilized as an input into a PCA function. In some embodiments, using PCA enables weighting of pre-processed signal datasets according to which pre-processed signal datasets have the most relevant information pertaining to cardiovascular parameters (e.g., cardiac motion) and to apply those weights to the pre-processed signal datasets to form a composite waveform.

In some embodiments, the PCA function outputs a set of principal components of the combined pre-processed signal datasets. One or more of the principal components (e.g., eigenvectors, as described herein) may be used to filter out noise from the pre-processed signal data in order to remove contributions not related to blood pressure, e.g., patient movement. In some embodiments, the set of principal components can include a first or a first set of principal components (e.g., PC1 components) that represent the highest amount of variability in the waveform data set. The first or first set of principal components can be used to represent the motion of a pulse (e.g., such as an arterial pulse). In some embodiments, the set of principal components can include higher order principal components (e.g., PC2 and PC3 components) that represent lower amounts of variability in the waveform dataset. The higher order principal components can be used to represent motion unrelated to a pulse (e.g., such as respiratory signals and/or motion artifacts).

In some embodiments, the PCA function performs an initial PCA computation S308 on the pre-processed signals, wherein such PCA computation may comprise computing PCA parameters that will be combined. In some embodiments, such PCA parameters comprise an eigenvector for each signal received within a given rolling window, wherein the eigenvector may correlate to a weight associated with the corresponding signal. As used herein, the terms “eigenvector” and “eigenvector value” may be used interchangeably. An eigenvector and eigenvector value may be examples of a component as described herein. Accordingly, in some cases, each channel of pre-processed signal data may be assigned a weight according to eigenvectors computed for each respective signal (of the channel). In some cases, the channels of radar data that provide the most signals are weighted more heavily than other channels of radar data.

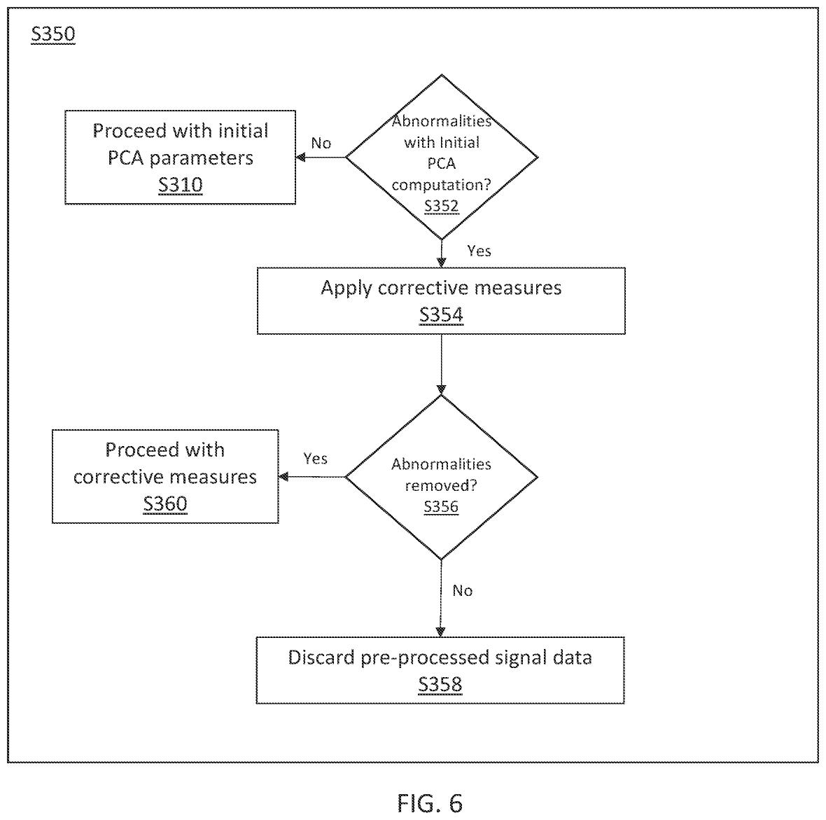

As described herein, in some embodiments, the system performs a quality assessment S350 on the initial PCA computation to validate the pre-processed signal data in a given rolling window. FIG. 6 depicts an exemplary flow chart for performing a quality assessment of the initial PCA computation. The system (e.g., via processor 170 as described herein) first identifies S352 any abnormalities with the initial PCA computation (e.g., using the eigenvectors as computed), such as abnormal drift, sign inversions (e.g., flips), and/or abnormal jumps or discontinuities. If no abnormalities are detected, then no change is made to either the rolling window size or the PCA parameters. By contrast, if abnormalities are detected with initial PCA computation, the system then applies corrective measures S354 to address such abnormalities. For example, if abnormal drift is detected with the initial PCA computations, then an exemplary corrective measure includes recomputing the rolling window size and/or performing drift correction. In some embodiments, drift can be corrected by evaluating the drift in view of models of expected blood pressure changes. In some embodiments, drift can be cancelled using sensor fusion to determine when drift is present and using morphological features of the waveform itself to determine when drift is present. In some embodiments, if, for example abnormal jumps or discontinuities are detected with the initial PCA computations, then an exemplary corrective measure includes filtering said jumps or discontinuities. Once the corrective measures have been applied S354, the system detects whether the abnormalities have been removed S356, such as removal of the abnormal drift and/or removal of the abnormal jumps or discontinuities. If the abnormalities remain, then the PCA parameters from the initial PCA computation are discarded S358. If the abnormalities have been removed, then the system proceeds with the corrective measures S360. For example, if the drift is found to be acceptable, then the system proceeds with the corrected PCA parameters, including filtered PCA parameters, and/or rolling window size. In some embodiments, corrective measures (e.g., drift correction, rolling window size adjustment, filtering of jumps/discontinuities) are applied on a feedback basis, such that the corrections are applied to a PCA computation for a signal data set in a subsequent rolling window, which are then evaluated for abnormalities per S352. In some embodiments, the corrective measures are applied in real-time, thereby correcting the initial PCA computation (e.g., eigenvectors) that had the original abnormalities.

In some embodiments, in addition to the quality assessment S350, the PCA function identifies other processing corrections to be made based on the initial PCA computation. For example, in some embodiments, the PCA function performs a sign correction S310 for a component (e.g., an eigenvector of a signal of a channel of data) if a sign flip is detected for the component's weight. For example, the PCA performs a sign correction S310 for a current eigenvector if its sign has changed (e.g., flipped or inverted) compared to a previous set of historical eigenvectors. In some embodiments, sign flip detection is performed for all components (e.g., eigenvectors). In some embodiments, sign flip detection is performed for a subset of the components (e.g., the component with the largest absolute weight, components with weights over a threshold magnitude, etc.) In some embodiments, if a sign flip is detected for the component with the largest absolute weight value, then sign correction is performed for the entire set of components (e.g., eigenvectors).

In some embodiments, sign flip detection for a component (e.g., eigenvector) comprises determining a first weight (for the component) for a first sample window (by performing an initial principal component analysis computation for pre-processed signal data of the first sample window (rolling window)), determining a second weight for subsequent component (e.g., eigenvector) for a subsequent sample window (by performing an initial principal component analysis computation for the pre-processed signal data of the second sample window), and comparing the first weight with the second weight to identify a sign flip. If a second weight for the subsequent component (e.g., eigenvector) has changed signs abruptly from the sign of the first weight, then sign correction is performed to revert the sign of the second weight back to the sign for the first weight. In some cases, sign ambiguity may be eliminated as described in “Resolving the Sign Ambiguity in the Singular Value Decomposition” by R. Bro, E. Acar, and T. Kolda, published by Sandia National Laboratories, and available at https://prod-ng.sandia.govitechlib-noauth/access-control.cgi/2007/076422.pdf. However, sign ambiguity or sign flips may be determined in any suitable manner.

With reference now to FIG. 3, in some embodiments, the PCA function then applies S312 the computed PCA parameters (e.g., eigenvectors for the pre-processed signals) to generate S314 a composite waveform data set based on the pre-processed signal data from the given rolling window. For example, in some embodiments, a linear combination of the signals from the data channel(s) with the weights given by the components (e.g., eigenvectors) is used to form the composite waveform dataset. In some embodiments, the first set of principal components (i.e. PC1 components) can provide the most relevant eigenvector for extracting a pulse waveform from pre-processed signal data (e.g., from the data channels). The first set of principal components may provide the most relevant eigenvector for extracting the pulse waveform based on the pulse motion being the dominant motion detected by a sensor (e.g., electromagnetic receiver 120). In some embodiments, the proper set of principal components for extracting the pulse motion can be selected from the PCA parameters by applying different sets of principal components (i.e. PC1, PC2, PC3, etc.) to the pre-processed signal data from a given rolling window. The resulting output waveform with the largest spectral content in the cardiac frequency range (e.g., 40 to 240 BPM) can indicate the proper set of principal components to be used to generate the composite waveform data set. Accordingly, in some embodiments, generating a waveform data set S314 functions to generate waveform data from a plurality of rolling windows of pre-processed signal data. Each data point in a waveform data set generated at S314 is computed from a plurality of pre-processed signal samples that are included in a given rolling window. As used herein, the terms “waveform data”, “composite waveform data”, “waveform data set”, and “composite waveform data set” may be used interchangeably.

With reference to FIG. 2, each waveform dataset generated is optionally post-processed S240, thereby generating a corresponding post-processed waveform data set. In some embodiments, performing waveform post processing comprises one or more of: filtering (any suitable type, such as bandpass filtering), pulse inversion correction, normalization, DC offset/drift correction, and others known in the art. In some cases, waveform filtering is adjusted to improve waveform data quality.

In some embodiments, the system (e.g., via processor 170) further performs a quality assessment S370 on one or more post-processed waveform data sets. In some embodiments, assessing the quality of waveform data (generated at S230) comprises identifying one or more of: pulse inversions, eigenvector sign flips, I/Q data that is abnormal or out of range, waveform features that are out of valid range (e.g., magnitude, timing, heart rate, etc.), and lack of signal. In some embodiments, other examples of abnormalities correlating with poor waveform quality can include power spectrum analysis and signal-to-noise ratio. In some embodiments, based on the quality assessment, a corrected post-processed waveform data set may be generated by applying one or more corrective measures. In some embodiments, where the post-processed waveform data set is not or cannot be corrected, said data set is then discarded and/or optionally alerted to a user.

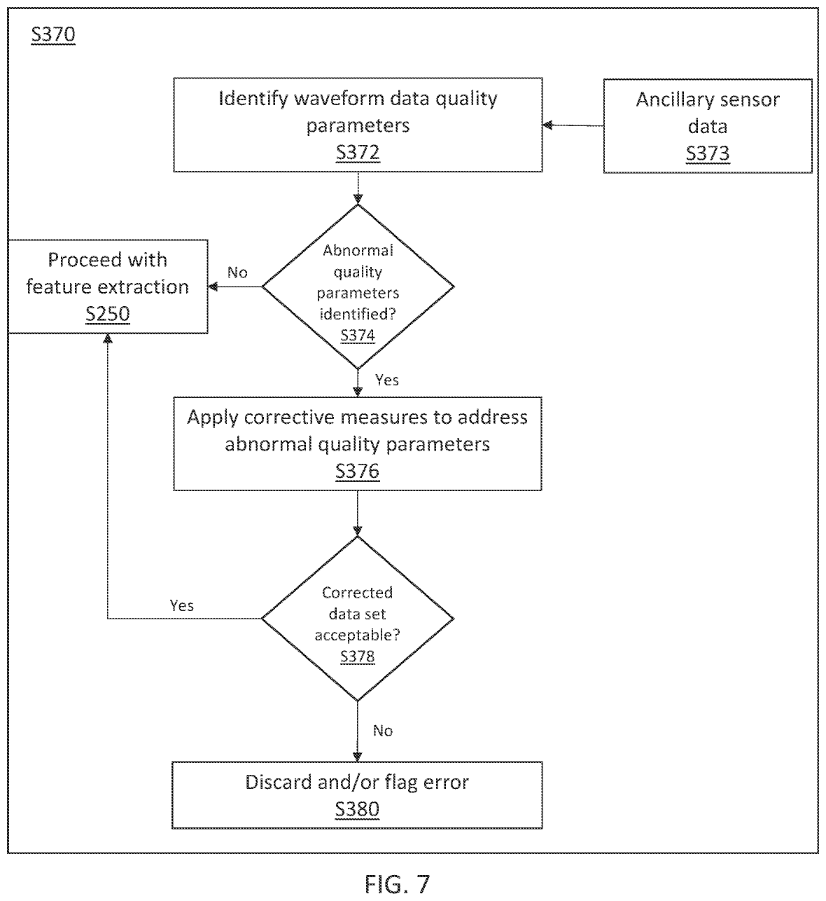

FIG. 7 provides an exemplary flow chart for performing a quality assessment S370 on a post-processed waveform data set generated (corresponding to a given rolling window). In some embodiments, if waveform data quality is poor, adjustments can be made to the post-processing applied to the waveform data generated. The system (e.g., processor 170 as described herein) first analyzes the post-processed waveform data set S372 to identify corresponding parameters correlating to a quality of the waveform data set. In some embodiments, the quality parameters comprise, for example, presence of abnormal drift in the waveform data set. In some embodiments, ancillary data S373 obtained from ancillary sensors are used to detect the quality of a post-processed waveform data set with consideration to one or more parameter as measured by the ancillary sensor(s). For example, in some embodiments the ancillary sensor(s) correlate to the motion of the user (sensed by using a motion sensor), temperature of the user, skin hydration level of the user, and/or reflectance and/or transmission of light through the skin of the user (e.g., thereby correlating to blood flow volume variations). In a specific example for motion of the user, in some embodiments, the quality parameters comprise the presence of excessive motion artifacts in the waveform data set.

If no abnormal quality parameters are detected with post-processed waveform data set, S374, and no other quality issue is detected, the post-processed waveform data set proceeds to feature extraction S250. By contrast, if an abnormal quality parameter is identified (e.g., abnormal drift and/or excessive motion artifacts), S374, then the system proceeds to apply corrective measures S376 to address such quality deficiencies with the post-processed waveform data set, and thereby generating a corrected post-processed waveform data set. Some non-limiting examples of quality parameters include magnitude, timing, and heart rate of the waveform data set. In some embodiments, such corrective measures include performing DC offset/drift correction, and/or performing waveform filtering to address abnormal drift. In some embodiments, such corrective measures include performing motion artifact removal and/or waveform reconstruction to address excessive motion artifacts in the waveform data set. In some cases, positioning of the health monitoring system 100 (or at least one component of the health monitoring system 100) is adjusted.

In some embodiments, the system again performs a quality assessment S378, this time on the corrected post-processed waveform data set. In some embodiments, the corrective measures are applied S376 in real time, such that the post-processed waveform data originally identified with quality issues is corrected. In some embodiments, the corrective measures are applied S376 in a feedback basis, such that post-processed waveform data in a subsequent rolling window is corrected. If the corrected post-processed waveform data set appears acceptable S378, the corrected post-processed waveform data set is then used for feature extraction S250. If however, the corrected post-processed waveform data set is still not acceptable, the corrected post-processed waveform data set is then discarded and/or the error is alerted to the user S380. For example, in some embodiments, the error is alerted the system via the processor 170, as described herein, and optionally using a display in communication with the processor (as described herein). In some embodiments, as described herein, the system includes a wearable device having a display in operative communication with the processor 170, and configured to display and/or alert the user or other operator regarding poor quality signals received. In some embodiments, the processor 170 is configured to communicate with an external device, such as a smart device (e.g., smart phone), or other device (e.g., a bedside monitor), relating to poor signal quality, wherein said external device is configured to alert the user or other operator.

As described herein, the system (e.g., via processor 170) is configured to perform one or more quality assessments on the signal data (pre-processed or post-processed) at multiple locations for the method 200 from FIG. 2 (e.g., see quality assessments S320, S330, S350, or S370). Accordingly, there are multiple safeguards to assess if the signal datasets begin to skew or deviate from an appropriate quality threshold. In some instances, if the quality assessment does not identify abnormalities with the raw data and/or transformed data (S320 S330 for example), any quality issues observed later on suggests other potential issues with the process, since the signals transmitted and received were acceptable. Other potential issues with the process, for example, can be rolling window size (too small or large). In some embodiments, the signal data can be analyzed to identify points of abnormalities, such as the identification of signal drift.

In some embodiments, extracting features from at least one waveform data set S250 functions to extract metrics from the waveform data set generated at S230 that are relevant to generating cardiovascular (e.g., blood pressure) data at S260. In some embodiments, such features may comprise one or more of: timing features, magnitude features, derivatives, area under curve, etc. In some embodiments, as described herein, machine learning algorithm(s) are used to identify pertinent features and trends that correlate with specific cardiovascular (e.g., blood pressure) data.

Generating cardiovascular (e.g., blood pressure) data at S260 may function to generate cardiovascular (e.g., blood pressure) data by processing the extracted features (extracted at S250) by using a cardiovascular (e.g., blood pressure) prediction model. In some cases, the cardiovascular prediction model may be or include a blood pressure prediction model. In some cases, the cardiovascular (e.g., blood pressure) data may comprise a continuous blood pressure signal that provides real-time blood pressure measurement of the user. In some embodiments, the cardiovascular (e.g., blood pressure) prediction model maps the extracted features to a blood pressure measurement. In some embodiments, a blood pressure cuff is used as a calibration input, so as to establish a baseline radar waveform correlating to a blood pressure measurement. Accordingly, in some embodiments, deviation from the baseline radar waveform can be correlated with changes from the calibrated blood pressure measurement.

In some embodiments, the cardiovascular (e.g., blood pressure) data is correlated with any one or more of: cardiovascular parameters, medical diagnoses, recommended treatments, respiratory parameters, tissue parameters, immune system parameters, digestive system parameters, endocrine system parameters, and/or any other suitable physiological parameters. Cardiovascular parameters can include any one or more of: blood pressure parameters (e.g., instantaneous blood pressure, blood pressure variability, mean arterial pressure, etc.), measures indicative of atherosclerosis or other cardiovascular disease, heartbeat parameters (e.g., instantaneous heart rate, heart rate variability, average heart rate, resting heart rate, heartbeat signature, etc.), pulse rate parameters (e.g., instantaneous pulse rate, pulse rate variability, etc.), physical activity parameters (e.g., motion metrics, fitness metrics, etc.), parameters correlated with cardiovascular-related health (e.g., sleep metrics, etc.), vital signs, pulse oximetry metric, measures of arterial stiffness, associated respiration parameters (e.g., respiratory rate, respiratory patterns, etc.), and/or any other suitable metric relating to cardiovascular-related health.

In some cases, generating cardiovascular (e.g., blood pressure) data may be performed by one or more of the methods described in U.S. Patent Publication Nos. 2020/0222011 or 2019/0282106, each of which is incorporated by reference herein in their entireties.

As described herein, in some embodiments, the method 200 comprises i) generating training data that may comprise features extracted from waveform data sets generated from received reflected electromagnetic signals from at least one user; and ii) training the cardiovascular (e.g., blood pressure) prediction model by using the generated training data.

In some embodiments, generated cardiovascular (e.g., blood pressure data may comprise one or more statistical analysis techniques or machine learning/artificial intelligence techniques. For example, in some embodiments, machine learning algorithms are used in order to make predictions using a set of input data, for example, a set of features extracted from waveform datasets. In some embodiments, machine learning algorithms comprise artificial neural networks (ANNs). In some embodiments, an artificial neural network is used by the system to process data, for example, to generate a cardiovascular (e.g., blood pressure) prediction model. For example, feedforward neural networks (such as convolutional neural networks or CNNs) and recurrent neural networks (RNNs) may be used. In some embodiments, neural networks are used to determine blood pressure by classifying data, for example, by associating a particular blood pressure based on collected features. In some cases, multiple layers of neural networks may be employed, creating a deep neural network. In some embodiments, using a deep neural network increases the predictive power of a neural network algorithm.

As described herein, in some embodiments, additional machine learning algorithms and statistical models are used in order to obtain insights from features extracted from the waveform datasets. In some embodiments, additional machine learning methods that are used may comprise logistic regressions, classification and regression tree algorithms, support vector machines (SVMs), naive Bayes, K-nearest neighbors, and random forest algorithms. In some embodiments, these algorithms are used for many different tasks, including data classification, clustering, density estimation, or dimensionality reduction. In some embodiments, machine learning algorithms are used for active learning, supervised learning, unsupervised learning, or semi-supervised learning tasks. In this disclosure, various statistical, machine learning, or deep learning algorithms may be used to predict a value of the blood pressure and/or to remove contributions from noise in the data set, e.g., user motion.

In some cases, contextual information may be used to determine whether to discard electromagnetic data samples received by the receiver 120. Such contextual information may be received via a sensor, user input device, external data source, etc. For example, contextual information may identify an activity performed by the user, and if such activity is likely to result in invalid blood pressure data (e.g., because of distorted signals, non-optimal physiological state, etc.), then the health monitoring system 100 may automatically discard data sampled during performance of such activity, thereby reducing the likelihood of invalid blood pressure data.

Embodiments of the system and/or method may comprise every combination and permutation of the various system components and the various method processes, wherein one or more instances of the method and/or processes described herein may be performed asynchronously (e.g., sequentially), contemporaneously (e.g., concurrently, in parallel, etc.), or in any other suitable order by and/or using one or more instances of the systems, elements, and/or entities described herein. Components and/or processes of the following system and/or method may be used with, in addition to, in lieu of, or otherwise integrated with all or a portion of the systems and/or methods disclosed in the applications mentioned above, each of which are incorporated in their entirety by this reference.

### 3. Computer Processor

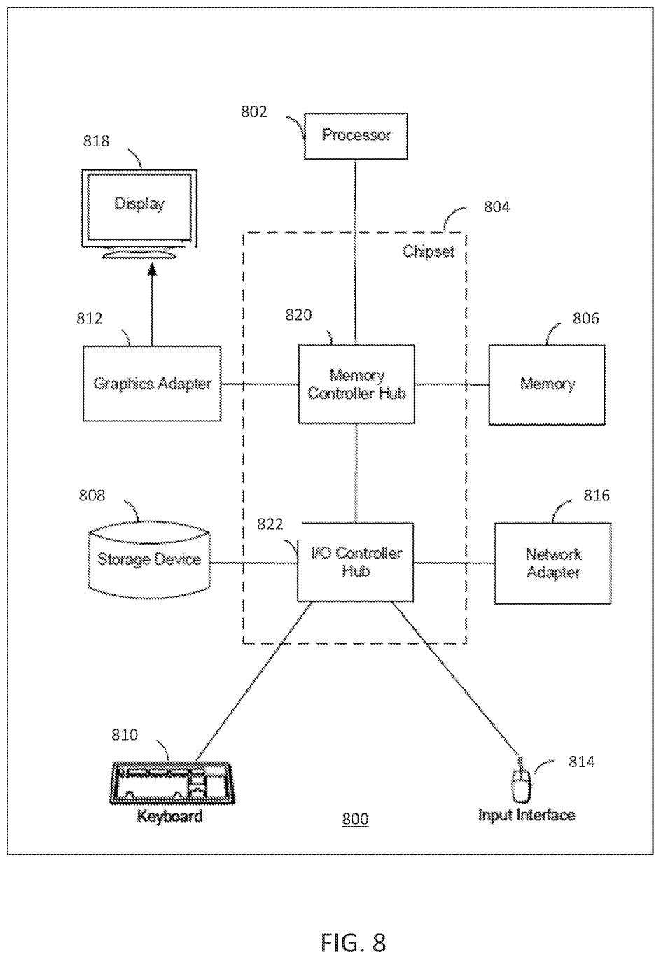

The present disclosure provides computer systems or computer processors that are programmed to implement methods of the disclosure. FIG. 8 shows a computer system 800 that is programmed or otherwise configured to generate blood pressure data and/or other cardiovascular data via waveform data from a radar system, as described herein. FIG. 8 may comprise an embodiment, variation, or example of processor 170 as disclosed herein. The computer system 800 can regulate various aspects of the methods of FIGS. 2-7 of the present disclosure, such as, for example, controlling an electromagnetic transmitter, controlling an electromagnetic receiver, receiving data from an electromagnetic receiver (and/or receiving IF data from a radar system), generating a waveform dataset, extracting features from a waveform data set, generating blood pressure data and/or other cardiovascular data, performing one or more quality assessments, etc. In some cases, the computer system 800 can be an electronic device of a user or a computer system that is remotely located with respect to the system of the present disclosure. The remote electronic device can be a mobile electronic device.

The computer 800 includes at least one processor 802 coupled to a chipset 804. The chipset 804 includes a memory controller hub 820 and an input/output (I/O) controller hub 822. A memory 806 and a graphics adapter 812 are coupled to the memory controller hub 820, and a display 818 is coupled to the graphics adapter 812. A storage device 808, an input device 814, and network adapter 816 are coupled to the I/O controller hub 822. Other embodiments of the computer 800 have different architectures.

The storage device 808 is a non-transitory computer-readable storage medium such as a hard drive, compact disk read-only memory (CD-ROM), DVD, or a solid-state memory device. The memory 806 holds instructions and data used by the processor 802. The input interface 814 is a touch-screen interface, a mouse, track ball, or other type of pointing device, a keyboard, or some combination thereof, and is used to input data into the computer 800. In some embodiments, the computer 800 may be configured to receive input (e.g., commands) from the input interface 814 via gestures from the user. The network adapter 816 couples the computer 800 to one or more computer networks.

The graphics adapter 812 displays images and other information on the display 818. In various embodiments, the display 818 is configured such that the user may input user selections on the display 818 to, for example, set one or more parameters of the radar system (e.g., rolling window size, use of transformed IF data, transmitter and/or receiver settings, etc.). In one embodiment, the display 818 may include a touch interface. In various embodiments, the display 818 can display blood pressure data and other cardiac health parameters, an indication to rest, an indication to raise or lower a limb, an indication that a blood pressure has reached an unhealthy level, an indication that a measurement is in progress, as well as any alerts relating to quality issues with the signal data (pre-processed or post-processed).

The computer 800 is adapted to execute computer program modules for providing functionality described herein. As used herein, the term “module” refers to computer program logic used to provide the specified functionality. Thus, a module can be implemented in hardware, firmware, and/or software. In one embodiment, program modules are stored on the storage device 808, loaded into the memory 806, and executed by the processor 802.

The types of computers 800 used by the entities of FIGS. 1-7 can vary depending upon the embodiment and the processing power required by the entity. For example, the radar system of system 100, for example, can run as a single processor 802 located on a wearable device, can run in a single computer 800 or multiple computers 800 communicating with each other through a network such as in a server farm. The computers 800 can lack some of the components described above, such as graphics adapters 812, and displays 818.

Methods and systems of the present disclosure can be implemented by way of one or more algorithms. An algorithm can be implemented by way of software upon execution by the processor 802. The algorithm can, for example, perform one or more steps of the methods of FIGS. 2-7, such as generating a waveform dataset, generating cardiovascular (e.g., blood pressure) data, generating a training dataset, performing one or more of the quality assessments, etc.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

1. A method for monitoring cardiovascular health using a wearable device, the method comprising:
(a) providing a wearable device comprising one or more RF electromagnetic transmitters, each configured to transmit an RF signal in the frequency range of 30-300 GHz towards a target body region on a subject, one or more RF electromagnetic receivers configured to receive reflected RF signals from the target body region, an attachment mechanism for fixing the one or more RF electromagnetic transmitters and receivers to the subject, and a standoff mechanism for fixing the one or more RF electromagnetic transmitters and receivers at a known stand-off distance from the target body region;
(b) transmitting one or more RF signals having a frequency in the range of 30-300 GHz towards a target body region of a subject, said target body region comprising skin over an artery in the subject;
(c) receiving one or more reflected RF signals corresponding to the reflection of the transmitted one or more RF signals from the target body region;
(d) generating a raw Intermediate Frequency (IF) signal by mixing or comparing the transmitted one or more RF signals and the reflected one or more RF signals, wherein the raw IF signal comprises signal data from one or more channels of the RF electromagnetic transmitter and the RF electromagnetic receiver;
(e) generating, via a processor, at least one waveform data set based on the raw or modified IF signal, wherein Dimensionality Reduction is used to combine signals from two or more channels to form a composite waveform;
(f) extracting one or more features from the composite waveform; and
(g) generating cardiovascular data from the features by using a cardiovascular model.

2. The method of claim 1, wherein the raw IF signal comprises any one, any two, any three, any four, any five, or any six of: timing, shape, phase, envelope, offset, and amplitude of the reflected RF signal.

3. The method of claim 1, where each channel comprises a signal measured by its own antenna and measurement circuit.

4. The method of claim 1, where the Dimensionality Reduction technique consists of Principal Component Analysis (PCA).

5. The method of claim 11, where the raw IF signal is transformed into the frequency domain to generate a frequency domain signal and magnitude, phase, real, and/or imaginary components of the frequency domain signal are utilized as separate inputs into the Principal Component Analysis.

6. The method of claim 12, where specific spectral regions of the frequency domain signal are selected that represent a spatial localization of the target body region.

7. The method of claim 12, wherein a window of IF data comprising multiple samples of data collected over a period of time is used to compute the PCA.

8. The method of claim 14, wherein the window is a rolling window.

9. The method of claim 14, where current PCA eigenvector values are periodically updated and historical PCA eigenvector values are stored and utilized to perform a quality assessment of current PCA eigenvector values, and/or future PCA eigenvector values.

10. The method of 16, the quality assessment includes determining if the current PCA eigenvector values have flipped sign compared to historical PCA eigenvector values, and provides an option for flipping the sign back in the current PCA eigenvector values.

11. The method of claim 16, where the quality assessment includes detection of noise or jumps in the current PCA eigenvector values and/or the historical PCA eigenvector values, and provides an option for filtering or removing the noise and jumps.

12. The method of claim 11, where a first set of principal components that represent the highest amount of variability in the data are used to represent the motion of a pulse such as an arterial pulse.

13. The method of claim 11, comprising selecting a principal component or first set of principal components (PCI) based on the largest output waveform amplitude swing with a frequency within a pulse rate range from 40 to 240 BPM.

14. The method of claim 13, where the higher order principal components (i.e. PC2, PC3) that represent lower amounts of variability in the data are used to represent motion unrelated to a pulse, including respiratory signals and or motion artifacts.

15. The method of claim 1, further comprising performing a first quality assessment on the raw IF signal, via the processor.

16. The method of claim 15, wherein the first quality assessment comprises:
a. detecting a first abnormality with the raw IF signal, and
b. applying a first corrective measure based on the first abnormality to form a corrected raw IF signal.

17. The method of claim 16, wherein the first quality assessment further comprises:
a. detecting the first abnormality or a different abnormality with the corrected raw IF signal, and
b. discarding the corrected raw IF signal and/or creating an alert based on the first abnormality or different abnormality.

18. The method of claim 16, wherein the first abnormality comprises signal saturation, a weak signal, a noisy signal, a small amplitude, or a combination thereof.

19. The method of claim 18, wherein the first corrective measure comprises i) reducing or increasing a strength of the transmitted electromagnetic signal, ii) adjusting the IF gain/filter settings, or iii) a combination thereof.

20. The method of claim 16, wherein the applying the first corrective measure is performed in real-time.
